(19) United States
(12) Patent Application Publication
(10) Pub. No.: US 2024/0288397 A1
(43) Pub. Date: Aug. 29, 2024

(54) CONDUCTOMETRIC SENSOR FOR DETECTING A NUCLEIC ACID AND A METHOD FOR THE DETECTION THEREOF

(71) Applicants: ROYAL MELBOURNE INSTITUTE OF TECHNOLOGY, Melbourne, Victoria (AU); PETER MACCALLUM CANCER INSTITUTE, Melbourne, Victoria (AU)

(72) Inventors: Taimur AHMED, Melbourne, Victoria (AU); Ganganath PERERA, Melbourne, Victoria (AU); Sumeet WALIA, Melbourne, Victoria (AU); Madhu BHASKARAN, Melbourne, Victoria (AU); Sharath SRIRAM, Melbourne, Victoria (AU); Stephen FOX, Melbourne, Victoria (AU); Andrew FELLOWES, Melbourne, Victoria (AU)

(21) Appl. No.: 18/572,665

(22) PCT Filed: Jun. 23, 2022
(86) PCT No.: PCT/AU2022/050637

§ 371 Date: Dec. 20, 2023

(30) Foreign Application Priority Data
Jun. 23, 2021 (AU) 2021901896

(51) Int. Cl. G01N 27/327

(52) U.S. Cl. CPC G01N 27/3275; G01N 27/3272

(57) ABSTRACT

The invention provides a sensor for detecting a nucleic acid, comprising: a substrate; a pair of terminal electrodes disposed on the substrate in mutually spaced apart and opposing relation; and a sensing element, between and in electrical contact with the pair of terminal electrodes, wherein the sensing element comprises: (i) a semiconducting portion of the substrate, wherein a conduction path between the terminal electrodes passes through the semiconducting portion; and (ii) an oligonucleotide on a surface of the semiconducting portion, the oligonucleotide being complementary to the nucleic acid to be detected, wherein hybridisation of the nucleic acid with the oligonucleotide leads to a change in resistance of the sensor.

### FIELD OF THE INVENTION

The present invention relates to sensors and, in particular, to a conductometric sensor for detecting a nucleic acid sequence in a fluid and a method for detecting a nucleic acid with such a sensor. The invention has been developed primarily for use in detecting nucleic acids in and from samples such as in a bodily fluid or tissues and will be described hereinafter with reference to this exemplary application.

The following discussion of the background to the invention is intended to facilitate an understanding of the invention. However, it should be appreciated that the discussion is not an acknowledgement or admission that any of the material referred to was published, known or part of the common general knowledge in Australia or any other country as at the priority date of any one of the claims of this specification.

### BACKGROUND OF THE INVENTION

The ability to accurately detect different nucleic acids of interest finds application in a wide range of fields of technology but is of particular importance in sensors used in medical diagnosis and treatment. For example, this finds application where the presence of specific nucleic acid sequences in samples obtained from individuals is indicative of a genetic mutation in the individual leading to them suffering from a disease such as cancer. This is particularly important where the difference between the native nucleic acids (e.g. RNA and subtypes, DNA, methylation types) and the nucleic acids of an individual suffering from the condition is a single point mutation in the nucleic acid and/or where the appropriate treatment is determined based on the changes in the nucleic acid in the individual.

An example of a condition of this type is melanoma with a BRAF V600E single nucleotide variant. Melanoma is a common skin cancer and is a global health consideration with a high rate of mortality when identified at late stage (˜1700 deaths per year in Australia). BRAF V600E is a common oncogenic point mutation in melanoma (˜40%) and the identification is needed for anti-BRAF targeted therapies. The ability to identify such subtle changes in nucleic acids is vital as certain treatments target the mutation whereas others are ineffective. Given the poor prognosis of patients with these medical conditions access to the correct therapy is vital to improve mortality rates. There are multiple other examples of changes/mutations in nucleic acids that are of diagnostic, prognostic and therapeutic importance.

Given the interest in the detection of specific nucleic acids, including the ability to detect mutations, there has been significant research in these areas. Accordingly, a wide range of conventional PCR-based techniques, allele-specific PCR, droplet digital PCR, high-resolution melting PCR, and PCR clamping and sequencing including Sanger and Next Generation Sequencing have been extensively used to detect mutations in DNA such as BRAF V600E in melanoma. Unfortunately, the conventional mutant DNA detection methods when used as cancer identification techniques heavily suffer from complexities in sample preparation, sample handling, operation, and data analysis. In addition, the tests are very time consuming, laborious, and expensive.

For example, the conventional BRAF V600E mutant DNA detection techniques include polymerase chain reaction (PCR)-based techniques, and sequence-based approaches including conventional Sanger and more recently Next Generation Sequencing.

These conventional methods are associated with at least the following common drawbacks (i) requirement of specific technical skills and knowledge, (ii) expensive bulky instrumentations and test costs (iii) extreme liability to sample contamination leading to false positive/negative results; (iv) complex and time-consuming sample preparation techniques (vary from few hours to days); and (v) requirement of careful control of experimental conditions in sample handling, preparation and operating of instrumentation.

As an alternative, optical sensors have been proposed for the detection of mutations including point mutations in nucleic acids and have utilized detection methods such as fluorescence spectroscopy, surface enhanced Raman spectroscopy, luminescence spectroscopy, and surface plasmon resonance spectroscopy. These optical sensors for point mutation detection in nucleic acid sequences suffer from the following limitations: (i) requirement of bulky instrumentation for the measurements (ii) requirement of suitable optically active tag/label to detect the biomarker of interest (eg: fluorescence spectroscopy); (iii) close overlap in optical bands of target biomarker and background components leading to lack of sensitivity; (iv) requirement of nanoparticles for the detection which may lead to nanoparticle cytotoxicity (eg: surface enhanced Raman spectroscopy and surface plasmon spectroscopy); (v) limitations in photostability and loss of recognition capability; and (vi) susceptibility to interference due to the factors such as pH, temperature, and oxygen levels

Electrochemical sensors have also previously been proposed for the detection of mutations including point mutations in nucleic acid sequences and have utilized detection methods such as amperometry, voltammetry (cyclic, square wave, and differential pulse), field effect transistors, and electrochemical impedance spectroscopy.

These electrochemical sensors for the detection of mutations including point mutation detection in nucleic acid sequences have the following disadvantages. (i) previous electrochemical methods require a complex detection technology (e.g.: electrochemical impedance spectroscopy and field effect transistors) whereas in this invention uses a straight forward conductometric detection method; (ii) electrochemical methods such as field effect transistors and cyclic voltammetry require a multiple number of electrodes leading to high power consumption; whereas this invention uses two terminal electrodes with low power consumption; (iii) some electrochemical methods utilize specific materials and complex sensor fabrication methods (e.g.: Nanomaterial associated-sensor platforms); (iv) some electrochemical techniques require specific enzymes (eg: endonuclease) for signal amplifications and specific redox couples (eg: amperometry) for signal generation; whereas this invention doesn't require those materials for detection.

Accordingly, notwithstanding the desirability to develop methods to enable detection of nucleic acids such as DNA sequences containing mutations there is still a requirement for further methods to achieve this outcome.

The present invention seeks to provide a sensor for use in detecting and/or quantifying the level of a nucleic acid in a sample and a method for detecting and/or quantifying the level of an altered nucleic acid, which will overcome or substantially ameliorate at least some of the deficiencies of the prior art, or to at least provide a useful alternative.

### SUMMARY OF THE INVENTION

According to a first aspect of the present invention, there is provided a sensor for detecting a nucleic acid, comprising: a substrate; a pair of terminal electrodes disposed on the substrate in mutually spaced apart and opposing relation; and a sensing element, between and in electrical contact with the pair of terminal electrodes, wherein the sensing element comprises: (i) a semiconducting portion of the substrate, wherein a conduction path between the terminal electrodes passes through the semiconducting portion; and (ii) an oligonucleotide on a surface of the semiconducting portion, the oligonucleotide being complementary to the nucleic acid to be detected, wherein hybridisation of the nucleic acid with the oligonucleotide leads to a change in resistance of the sensor.

The semiconducting portion of the substrate that forms part of the sensing element may take many forms. In one embodiment the semiconducting portion comprises a high resistivity non-oxide semiconductor. In one embodiment the semiconducting portion comprises an oxygen-deficient metal oxide.

In some embodiments, the semiconducting portion has a resistivity of greater than 100 ohm·cm. In some embodiments, the semiconducting portion has a resistivity of greater than 200 ohm·cm. In some embodiments, the semiconducting portion has a resistivity of greater than 500 ohm·cm. In some embodiments, the semiconducting portion has a resistivity of greater than 1000 ohm·cm. In some embodiments, the semiconducting portion has a resistivity of greater than 2000 ohm·cm. In some embodiments, the semiconducting portion has a resistivity of greater than 5000 ohm·cm.

In some embodiments, semiconducting portion has a resistivity in the range of about 500 ohm·cm to about 5000,000 ohm·cm. In some embodiments, semiconducting portion has a resistivity in the range of about 1000 ohm·cm to about 5000,000 ohm·cm. In some embodiments, semiconducting portion has a resistivity in the range of about 5000 ohm·cm to about 5000,000 ohm·cm. In some embodiments, semiconducting portion has a resistivity in the range of about 1000 ohm·cm to about 10000 ohm·cm.

In some embodiments the semiconducting portion comprises a high resistivity non-oxide semiconductor. In some embodiments, the non-oxide semiconductor has a resistivity of greater than 100 ohm·cm. In some embodiments, the non-oxide semiconductor has a resistivity in the range of about 500 ohm·cm to about 50,000 ohm·cm, or in the range of about 1000 ohm·cm to about 10000 ohm·cm

In some embodiments, the sensor has an electrical resistance in the range of about 10 kiloohms to about 10000 kiloohms.

In some embodiments, the non-oxide semiconductor is selected from the group consisting of an elemental semiconductor and a compound semiconductor. In some embodiments, the non-oxide semiconductor is an elemental semiconductor.

In some embodiments, the non-oxide semiconductor is a silicon semiconductor. The silicon semiconductor may be an intrinsic silicon semiconductor. The silicon semiconductor may be a float-zone silicon semiconductor.

In some embodiments, the substrate comprises the semiconducting portion as an integral portion thereof. The substrate may be a wafer of the non-oxide semiconductor.

In another embodiment the semiconducting portion comprises an oxygen-deficient metal oxide. In some embodiments the oxygen-deficient metal oxide has a resistivity in the range of about 500 ohm·cm to about 5000,000 ohm·cm. In some embodiments the oxygen-deficient metal oxide has a resistivity in the range of about 1000 ohm·cm to about 5000,000 ohm·cm. In some embodiments the oxygen-deficient metal oxide has a resistivity in the range of about 5000 ohm·cm to about 5000,000 ohm·cm. In some embodiments the oxygen-deficient metal oxide has a resistivity in the range of about 1000 ohm·cm to about 10000 ohm·cm.

In some embodiments the oxygen-deficient metal oxide is selected from the group consisting of zinc oxide (ZnO), strontium titanium oxide (STO), tin oxide (SnO₂), and titanium dioxide (TiO₂). In some embodiments the oxygen-deficient metal oxide layer is oxygen deficient zinc oxide.

In some embodiments, the oligonucleotide is chemically bonded to the semiconducting portion, for example by an organic linker which may be the residue of a silanizing agent. The oligonucleotide may be chemically bonded to the semiconducting layer by a process comprising: (i) silanization of the non-oxide semiconductor with a silanizing agent having a terminal functionality selected from the group consisting of an epoxy group, a thiol group, an amino group, a carboxy group and a hydroxy group, and (ii) reacting a precursor comprising the oligonucleotide with the terminal functionality. The silanizing agent is selected from the group consisting of (3-glycidyloxypropyl) trimethoxysilane (GPS), (3-mercaptopropyl) trimethoxysilane (MTS), (3-aminopropyl) triethoxysilane (APTES), and N-(2-aminoethyl)-3-aminopropyl-trimethoxysilane (AEAPTS).

In some embodiments the oligonucleotide is complementary to a nucleic acid having a single point mutation to a native DNA sequence. In some embodiments the oligonucleotide is complementary to a nucleic acid having an insertion or deletion mutation relative to a native DNA sequence. In some embodiments the oligonucleotide is attached to the surface of the semiconducting portion via the 3′ end of the oligonucleotide. In some embodiments the oligonucleotide is attached to the surface of the semiconducting portion via the 5′ end of the oligonucleotide. In some embodiments the oligonucleotide contains at least one 2′-O,4′-C-methylene-β-D-ribofuranosyl nucleotide monomeric unit i.e a locked nucleic acid (LNA) (.Vester, B., Wengel, J., LNA (locked nucleic acid): high-affinity targeting of complementary RNA and DNA. Biochemistry 43, 13233-13241 (2004).

In some embodiments the oligonucleotide is complementary to sequence from the human BRAF gene. In some embodiments the oligonucleotide is complementary to a microRNA sequence. In some embodiments the oligonucleotide is complementary to variants of a pharmacogenetic gene.

The sensor is suitably a conductometric sensor. The sensor may thus comprise apparatus to apply a voltage between the terminal electrodes and to measure the current flow through the conduction path of the sensor. The apparatus may suitably be a potentiostat. In embodiments, therefore, the sensor is not a field effect transistor

According to a second aspect of the present invention, there is provided a method for detecting a nucleic acid, the method comprising the steps of (a) contacting a sensing element of a sensor of the invention with a substance possibly containing the nucleic acid; (b) measuring an electrochemical parameter of the sensor corresponding to a resistance of the sensor; and (c) detecting the presence or absence of the nucleic acid on the sensing element based on electrochemical parameter measured in step (b).

The applicants have found that hybridisation of a nucleic acid with the oligonucleotide leads to change in resistance of the sensor. Without wishing to be bound by theory it is felt that this is due to a change in electrical environment caused by hybridisation of the target nucleic acid to the oligonucleotide. There are a number of different parameters that may be measured. In one embodiment measuring an electrochemical parameter of the sensor comprises: (i) applying a voltage across the sensor; and (ii) measuring a current flow through the sensor.

In some embodiments, measuring an electrochemical parameter of the sensor comprises: (i) applying a voltage across the sensor; and (ii) measuring a current flow through the sensor.

In some embodiments, detecting the presence or absence of the nucleic acid comprises comparing the electrochemical parameter measured in step b) with a reference value for that parameter for the sensor. In some embodiments an increase in resistance of the sensor relative to the reference resistance for the sensor is indicative of the presence of the nucleic acid on the sensor and hence in the sample.

In some embodiments, the substance is a sample solution, from nucleic acids extracted from tissues and/or from a bodily fluid.

According to a third aspect of the present invention, there is provided a method of fabricating a sensor for detecting a nucleic acid the method comprising the steps of: providing a substrate comprising a semiconducting portion; producing a pair of terminal electrodes on the substrate in mutually spaced apart and opposing relation, wherein the semiconducting portion of the substrate is positioned between and in electrical contact with the terminal electrodes and wherein a conduction path between the terminal electrodes passes through the semiconducting portion; and immobilising an oligonucleotide on a surface of the semiconducting portion, the oligonucleotide being complementary to the nucleic acid to be detected, thereby producing a sensing element comprising (i) the semiconducting portion and (ii) the oligonucleotide.

Other aspects of the invention are also disclosed.

### BRIEF DESCRIPTION OF THE DRAWINGS

Notwithstanding any other forms which may fall within the scope of the present invention, preferred embodiments of the invention will now be described, by way of example only, with reference to the accompanying drawings in which:

FIG. 1 shows a schematic representation of one embodiment of a conductometric sensor for detecting a nucleic acid in accordance with certain embodiments of the present invention, in which the sensor has a sensing element comprising a semiconducting portion of the sensor substrate with an oligonucleotide immobilised on the surface of the semiconducting portion, the oligonucleotide being complementary to the nucleic acid sequence to be detected.

FIG. 2 shows a schematic representation of a method for fabricating the conductometric sensor depicted in FIG. 1.

FIG. 3 shows a schematic representation of another embodiment of a conductometric sensor for detecting a nucleic acid in accordance with certain embodiments of the present invention, in which the sensor has a sensing element comprising a semiconducting portion of the sensor substrate with an oligonucleotide immobilised on the surface of the semiconducting portion, the oligonucleotide being complementary to the nucleic acid sequence to be detected.

FIG. 4 shows a schematic representation of a method for fabricating the conductometric sensor depicted in FIG. 3.

FIG. 5 shows a schematic representation of sensor formation and nucleic acid detection on a high resistivity non oxide semiconductor.

FIG. 6 shows a schematic representation of sensor formation and nucleic acid sequence detection on an oxygen-deficient metal oxide.

FIG. 7 shows a graphical representation of the detection of single point mutated DNA on (a) 3′ and (b) 5′ amine immobilised oligonucleotides on a metal oxide (ZnO₍₁₋ₓ₎ sensor).

FIG. 8 shows a graphical representation a selectivity study for mutated DNA on (a) 3′ and (b) 5′ amine immobilised oligonucleotides on a metal oxide (ZnO₍₁₋ₓ₎ sensor).

FIG. 9 shows a graphical representation of the detection of single point mutated DNA on 5′ amine immobilised oligonucleotides on a Silicon sensor.

### DETAILED DESCRIPTION OF SPECIFIC EMBODIMENTS

### Summary of Sequence Identifiers

| Sequence Identifier | Description |
|---|---|
| SEQ ID NO: 1 | BRAF 3′-amine DNA oligonucleotide |
| SEQ ID NO. 2 | BRAF 5′-amine DNA oligonucleotide |

BRAF 3′-amine DNA oligonucleotide
(SEQ ID NO. 1)
(5′-GGTCTAGCTACAGAGAAATCTCGAT/3AmMO/-3′)
BRAF 5′-amine DNA oligonucleotide
(SEQ ID NO. 2)
(5′-/AmMC6/GGTCTAGCTACAGAGAAATCTCGAT-3′)

The present invention relates to a conductometric sensor for detecting a nucleic acid. The sensor comprises a substrate, a pair of terminal electrodes disposed on the substrate in mutually spaced apart and opposing relation, and a sensing element located between and in electrical contact with the pair of terminal electrodes. The sensing element comprises: (i) a semiconducting portion of the substrate and (ii) an oligonucleotide or oligonucleotides on a surface of the semiconducting portion, the oligonucleotide(s) being complementary to the nucleic acid(s) to be detected. An electrical conduction path between the terminal electrodes passes through the semiconducting portion. In use, binding of a nucleic acid sequence to the oligonucleotide causes a change in electrical resistance of the sensor. Without wishing to be bound by theory it is felt that the binding of the nucleic acid sequence to the oligonucleotide leads to a change in electron density on the sensor which in turn leads to a change in resistance of the sensor. Accordingly, the increase in resistance can be determined by measuring an electrochemical parameter of the sensor corresponding to the resistance of the sensor. For example, the resistance you can measure the current response when a voltage is applied across the sensor, and the presence, absence and/or concentration of the nucleic acid can thus be detected. In one embodiment the presence of the nucleic acid of interest in the sample is indicated by an increase in resistance of the sensor following incubation of the sensor with the sample. In yet another embodiment the presence of the nucleic acid of interest in the sample is indicated by an increase in resistance of the sensor following incubation of the sensor with the sample whereas incubation with a native sequence led to decreased resistance.

The sensor of the present invention thus employs a conductometric sensing technique for detecting a range of potential nucleic acids in samples such as in a fluid. Examples of exemplary fluids include bodily fluid such as human saliva, blood, plasma, interstitial fluid, cerebrospinal fluid, tears and/or sweat, or extracted nucleic acids from tissues for the prognosis/diagnosis/therapy for a medical conditions or for other characteristics of an individual person and for pharmacogenomics. The sensors can also be used to detect nucleic acids in gaseous samples such as in respiratory aerosol droplets or in ventilation systems. As will be described in more detail below, the conductometric sensor has a simple and comparatively easy-to-fabricate device structure, which offers a cost-effective alternative to conventional non-invasive sensors that either require specialized substrates or adopt sensing techniques that limit their accuracy.

What follows is a detailed description of the non-invasive conductometric sensor and a method for the application thereof for detecting the levels of a range of nucleic acids in a sample such as in a fluid. It should be noted in the following description that like or the same reference numerals in different embodiments denote the same or similar features.

### Sensor

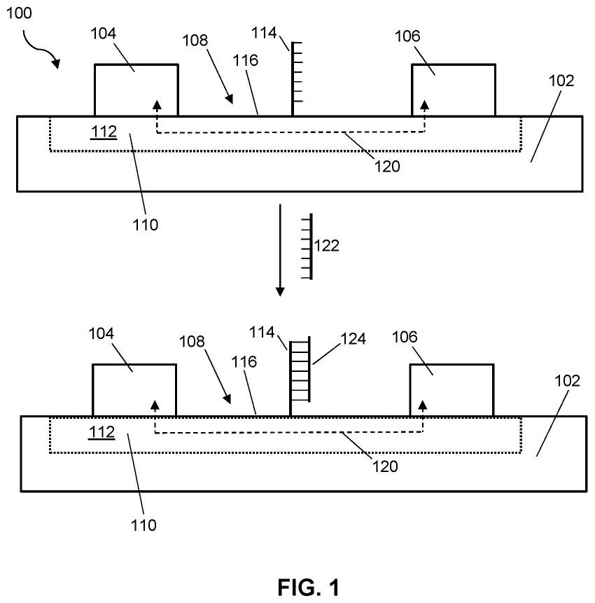

In one form, and as shown in the schematic representation in FIG. 1, a sensor 100 comprises a substrate 102, a pair of terminal electrodes 104, 106 disposed on the substrate in mutually spaced apart and opposing relation, and a sensing element 108, between and in electrical contact with terminal electrodes 104, 106. Sensing element 108 comprises a semiconducting portion 110 which comprises a semiconducting material 112, and an oligonucleotide 114 on surface 116 of semiconducting portion 110. A conduction path 120 between terminal electrodes 104 and 106 passes through semiconducting portion 110, and thus through the semiconducting material 112.

In the embodiment shown in FIG. 1, substrate 102 comprises semiconducting portion 110 as an integral part of the substrate, and the remainder of the substrate is thus composed of the same semiconducting material 112. The conductive pathway 120 between terminals 104 and 106 is substantially confined to a surface layer of the substrate (corresponding to semiconducting portion 110) by the electric field lines established when a voltage in applied across the sensor in use. Advantageously, there is thus no need to fabricate a discrete thin-film semiconducting layer on the sensor substrate. Substrate 102 may thus be of any convenient thickness, for example as provided when using a wafer of the semiconducting material 112.

Alternatively, sensing element 108 may include semiconducting portion 110 formed as a discrete, surface layer on substrate 102, at least between terminal electrodes 104 and 106 but optionally extending across the entire substrate surface. In such embodiments, substrate 102 may be composed of any suitable material capable of receiving and supporting semiconducting layer 110.

In use, sensor 100 is contacted with a substance, such as sample solution 122, which contains (or may contain) nucleic acid 124. The nucleic acid, when present, hybridises with oligonucleotide 114, thereby causing a change in electrical resistance of the sensor. The change in electrical resistance occurs due to charge transfer when the incoming complementary DNA strand is hybridized with the oligonucleotide, by donating electrons to or accepting electrons from the semiconductor. When a voltage is applied across the sensor, i.e. between terminal electrodes 104 and 106, the resultant current flowing between the terminal electrodes along conductive pathway 120 can be measured and the electrical resistance of the sensor thus determined. By comparing this resistance with a predefined reference resistance for the sensor, the presence or absence of the nucleic in sample solution 122 may be detected.

As the skilled person will appreciate, sensing element 108 will typically contain a plurality of oligonucleotides 114 and the fraction of those oligonucleotides which are hybridised with 124 may depend on the nucleic concentration in sample solution 122. Because the resistance of conductive pathway 120 will be proportionate to the fraction of hybridised oligonucleotide sites 114, the concentration of nucleic acid 124 in fluid 122 may thus be determined, for example by comparing the resistance as determined with a calibration curve.

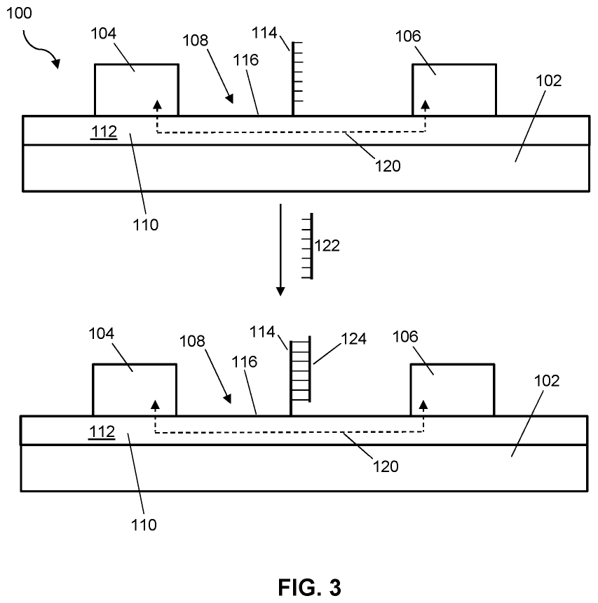

In another form, and as shown in the schematic representation in FIG. 3, a sensor 100 comprises a substrate 102, a pair of terminal electrodes 104, 106 disposed on the substrate in mutually spaced apart and opposing relation, and a sensing element 108, between and in electrical contact with terminal electrodes 104, 106. Sensing element 108 comprises a semiconducting portion 110 which comprises semiconducting layer 112 on the underlying substrate and an oligonucleotide 114 on surface 116 of semiconducting portion 110. A conduction path 120 between terminal electrodes 104 and 106 passes through semiconducting portion 110, and thus through the semiconducting material 112.

In the embodiment shown in FIG. 3, substrate 102 comprises semiconducting portion 110 as a layer portion of the substrate, and the remainder of the substrate is thus composed of a support material. The conductive pathway 120 between terminals 104 and 106 is substantially confined to the layer corresponding to semiconducting portion 110.

Accordingly, in this embodiment, sensing element 108 includes a semiconducting portion 110 formed as a discrete, surface layer on substrate 102, at least between terminal electrodes 104 and 106 but optionally extending across the entire substrate surface. In such embodiments, substrate 102 may be composed of any suitable material capable of receiving and supporting semiconducting layer 110.

In use, sensor 100 is contacted with a substance, such as sample solution 122, which contains (or may contain) nucleic acid 124. The nucleic acid, when present, binds to oligonucleotide 114, thereby causing a change in electrical resistance of the sensor. The change in electrical resistance occurs due to charge transfer when the incoming complementary DNA strand is hybridized with the oligonucleotide, by donating electrons to or accepting electrons from the semiconductor. When a voltage is applied across the sensor, i.e. between terminal electrodes 104 and 106, the resultant current flowing between the terminal electrodes along conductive pathway 120 can be measured and the electrical resistance of the sensor thus determined. By comparing this resistance with a predefined reference resistance for the sensor, the presence or absence of the oligonucleotide in sample solution 122 may be detected.

As before, the skilled person will appreciate, that sensing element 108 will typically contain a plurality of oligonucleotide sites 114 and the fraction of those binding sites which are hybridised with nucleic acid 124 may depend on the nucleic acid concentration in sample solution 122. Because the resistance of conductive pathway 120 will be proportionate to the fraction of hybridised oligonucleotides 114, the concentration of nucleic acid 124 in fluid 122 may thus be determined, for example by comparing the resistance as determined with a calibration curve.

What follows is a description of each of the components of the conductometric sensor.

### Substrate

In the broadest form of the invention, the substrate as a whole is not particularly limited and may for example be manufactured from a material selected from the group consisting of a semiconductor, a polymer, a glass or a ceramic.

For instance, suitable polymers for use as the substrate may be selected from the group consisting of polydimethylsiloxane (PDMS), polyimide (PI) and polyethylene naphthalate (PEN). While suitable ceramics may be selected from the group consisting of aluminium oxide (Al₂O₃), sapphire and silicon nitride (Si₃N₄).

As previously stated at least a portion of substrate comprises a semiconducting portion which forms part of the sensing element. In certain embodiments the semiconducting portion represents the entire substrate. In certain embodiments the semiconducting portion represents only a part of the substrate as a whole. In such embodiments, the semiconducting portion of the sensing element may be supported on a support layer of the substrate, optionally only in the substrate region covered by the sensing element. In certain embodiments the semiconducting portion of the substrate forming the sensing element is in the form of a layer on one side of the support layer of the substrate. In certain embodiments the semiconducting portion of the substrate forming the sensing element is in the form of a strip on, or indented in, the support layer of the substrate between the electrodes. In some preferred embodiments, however, the substrate comprises, or consists of, the semiconducting portion. In this embodiment the semiconducting portion of the substrate represents the entire substrate. As seen in FIG. 1, the semiconducting portion of the sensing element may thus be an integral portion of the substrate, simplifying the overall device architecture. In some embodiments, the substrate is a wafer of a semiconducting material.

### Electrodes

The sensor comprises a pair of terminal electrodes disposed on the substrate in mutually spaced apart and opposing relation. The sensing element of the sensor is thus located in a sensing region between the spaced apart terminal electrodes. As will be apparent to the skilled person, the terminal electrodes are electrically conductive and configured for electrical connection to an apparatus for applying a voltage across the sensor, such as a potentiostat.

As shown in FIG. 1, the terminal electrodes are formed as discrete structures on top of the substrate surface and in electrical contact with the underlying semiconducting portion of the substrate. However, other configurations are also envisaged. For example, the terminal electrodes may be recessed into the substrate, with the semiconducting portion of the sensing element lying horizontally between the terminal electrodes along the substrate surface.

The terminal electrodes may comprise a conductive metal or alloy, preferably a metal or alloy which is chemically inert. Gold is one example of a suitable metal.

In some embodiments, terminal electrodes are formed on the substrate by microfabrication techniques. Gold terminal electrodes may be formed by evaporating a gold thin film (250 nm with 100 nm chromium adhesion layer) onto the semiconducting layer using electron beam lithography. The as deposited gold thin film is then patterned using standard photolithography and wet etching techniques to define the pair of terminal electrodes.

The terminal electrodes may generally be sized and arranged relative to each other in any suitable configuration for a conductometric sensor. In some embodiments, the terminal electrodes are spaced apart by a distance in the range of 1 micrometer to 100 micrometer. In some embodiments, the terminal electrodes have a length (i.e. in a direction orthogonal to the inter-electrode gap distance) in the range of 200 to 4000 micrometer. The inventors have obtained good results using two parallel electrodes of 4000 micrometers length, spaced apart by 40 micrometers, thus providing a sensing region having an area of 16×10⁻⁸ m².

In principle the sensor will work with a wide variety of sensor geometry and dimensions in relation to the electrodes. Nevertheless, the electrodes are typically spaced from 1 μm to 200 μm apart. In one embodiment the electrodes are spaced from 1 μm to 100 μm apart. In one embodiment the electrodes are spaced from 10 μm to 80 μm apart. In one embodiment the electrodes are spaced from 20 μm to 60 μm apart. In one embodiment the electrodes are spaced from 30 μm to 50 μm apart. In one embodiment the electrodes are spaced from 35 μm to 45 μm apart. In one embodiment the electrodes are spaced about 40 μm apart.

The sensing electrodes can also vary in width with the width typically being from 200 to 4000 μm wide. In one embodiment the sensing electrodes are from 400 to 3000 μm wide. In one embodiment the sensing electrodes are from 800 to 2000 μm wide. In one embodiment the sensing electrodes are from 1000 to 1500 μm wide.

### Sensing Element

The sensor comprises a sensing element which comprises (i) a semiconducting portion of the substrate and (ii) an oligonucleotide(s) on a surface of the semiconducting portion, the oligonucleotide(s) being complementary to the nucleic acid(s) to be detected. As will be appreciated by a skilled worker in the art the sensor may be fabricated with a single type of oligonucleotide on a surface or it may be fabricated with a plurality of different oligonucleotides depending upon the end use application. In one embodiment the sensor is fabricated with a single oligonucleotide. In one embodiment the sensor is fabricated with a plurality of different oligonucleotides. As will also be appreciate the sensor is more sensitive if there is only one type of oligonucleotide on the surface as this reduces the possibility of interference between the different oligonucleotides. Nevertheless, in principle, the sensor may contain a number of different oligonucleotides such that a plurality of different nucleic acids can be detected with a single test. The nucleic acid can be of any class and type.

The sensing element is located between the terminal electrodes and is in electrical contact with both terminal electrodes. The device is thus configured so that the electrical conduction path between the terminal electrodes passes through the semiconducting portion, and thus through the semiconducting portion.

### Semiconducting Portion

In some embodiments the semiconducting portion is an integral portion of the substrate, in particular a region or surface portion of the substrate which extends across the sensing region between the terminal electrodes.

In other embodiments, the semiconducting portion is a discrete surface layer of the substrate which is supported on an underlying support layer of the substrate. The semiconducting layer is located at least in the sensing region between the terminal electrodes, but may optionally extend across the entire substrate surface. In such embodiments, the terminal electrodes may be formed, for example by metal deposition, on the surface of the discrete semiconducting layer of the substrate. Alternatively, the terminal electrodes may be formed on the support layer, and the semiconducting portion of the substrate is subsequently formed on the support layer of the substrate in at least the sensing region between the terminal electrodes.

The semiconducting portion of the substrate may take a number of different forms. In one embodiment the semiconducting portion comprises, and typically consists of, a high-resistivity non-oxide semiconductor. As used herein, a non-oxide semiconductor includes both elemental semiconductor materials and compound semiconductor materials, but excludes metal oxide semiconductors. In another embodiment the semiconducting portion comprises an oxygen-deficient metal oxide.

Common semiconductors used in electrochemical devices, including many non-oxide semiconductors such as doped silicon, are too conductive for use in a conductometric sensing element. Any effect on the electronic properties of such semiconductors caused by oligonucleotides binding on the surface will be too small to provide sufficient sensitivity. For this reason, previous conductometric sensors have typically been constructed with a discrete conductometric sensing layer of a high-resistivity polymeric or metal oxide material.

The applicants have surprisingly found that in order to be able to provide a suitably sensitive sensor that is capable of detecting the sometimes very minor changes in electrical environment caused by hybridisation of a nucleic acid sequence to a pendant oligonucleotide bound to the surface of the sensor the semiconducting material used must have quite high resistivity. In some embodiments, the semiconductor material has a resistivity of greater than 100 ohm·cm, or greater than 200 ohm·cm. Resistivities of this type or higher are found to provide suitable sensitivity to the sensor in detecting bound oligonucleotides.

In some embodiments, the semiconducting portion has a resistivity of greater than 100 ohm·cm. In some embodiments, the semiconducting portion has a resistivity of greater than 200 ohm·cm. In some embodiments, the semiconducting portion has a resistivity of greater than 500 ohm·cm. In some embodiments, the semiconducting portion has a resistivity of greater than 1000 ohm·cm. In some embodiments, the semiconducting portion has a resistivity of greater than 2000 ohm·cm. In some embodiments, the semiconducting portion has a resistivity of greater than 5000 ohm·cm.

In some embodiments, semiconducting portion has a resistivity in the range of about 500 ohm·cm to about 5000,000 ohm·cm. In some embodiments, semiconducting portion has a resistivity in the range of about 1000 ohm·cm to about 5000,000 ohm·cm. In some embodiments, semiconducting portion has a resistivity in the range of about 5000 ohm·cm to about 5000,000 ohm·cm. In some embodiments, semiconducting portion has a resistivity in the range of about 1000 ohm·cm to about 10000 ohm·cm.

Surprisingly, therefore, it has now been found that good conductometric sensor performance can be obtained when using a high-resistivity non-oxide semiconductor for the conductometric sensing element. By selecting a non-oxide semiconductor with high resistivity, the sensor has an overall resistance which falls in a range suitable for the detection of changes in electrical environment caused by hybridisation of a nucleic acid sequence to a pendant oligonucleotide bound at the sensing element surface.

In some embodiments, the high-resistivity non-oxide semiconductor has a resistivity of greater than 100 ohm·cm, or greater than 200 ohm·cm. By contrast, doped silicon semiconductors commonly used in electrochemical sensing devices generally have a resistivity of from about 1 to 10 ohm·cm.

In some embodiments, the high-resistivity non-oxide semiconductor has a resistivity in the range of 500 ohm·cm to about 50,000 ohm·cm, such as in the range of about 1000 ohm·cm to about 10000 ohm·cm. The inventors have obtained good results with non-oxide semiconductors having resistivities of 1000 ohm·cm and 5000-10000 ohm·cm.

The high-resistivity non-oxide semiconductor may be selected so that the sensor has a suitable electrical resistance, as measured between the terminal electrodes (and along the conduction path). In some embodiments, the sensor has an electrical resistance in the range of about 10 kiloohms to about 10000 kiloohms, for example in the absence of any nucleic acid sequence being hybridised with the oligonucleotide. The inventors have found that very poor sensitivity to bioanalytes is obtained when low resistance sensors are used.

In some embodiments, the non-oxide semiconductor is selected from the group consisting of an elemental semiconductor and a compound semiconductor.

Suitable elemental semiconductors may include silicon and germanium semiconductors, preferably silicon semiconductors. High purity intrinsic (undoped) silicon semiconductors have been found particularly suitable due to their resistive properties. The intrinsic silicon semiconductor may be a float zone silicon, which is a high purity silicon prepared by the float zone refining technique. In this technique, a molten region is slowly passed along a rod of silicon with the impurities preferentially remaining in the molten region instead of being reincorporated into the recrystallised silicon. By contrast, most silicon semiconductor is produced by the Czochralski process and thus incorporates a higher degree of impurities. A suitable float zone silicon are 3″ and 4″ wafers with <100> orientation.

While intrinsic silicon semiconductors have been found particularly suitable, it is not excluded that the non-oxide semiconductor may be a doped elemental semiconductor, provided that the level of doping is sufficiently low that the semiconductor remains highly resistive.

Suitable compound semiconductors may include binary semiconductors such as gallium arsenide (GaAs), indium phosphide (InP) and indium antimonide (InSb), ternary semiconductors such as gallium aluminium arsenide (GaAlAs), and the like.

As already noted, in certain embodiments the semiconducting portion comprising high-resistivity non-oxide semiconductor may be an integral portion of the substrate, and the substrate may thus comprise, or consist of the non-oxide semiconductor. For example, the substrate may be a wafer of the non-oxide semiconductor, such as a wafer of high-resistivity intrinsic silicon semiconductor.

In yet an even further embodiment the semiconducting portion comprises an oxygen-deficient metal oxide. In some embodiments the oxygen-deficient metal oxide has a resistivity in the range of about 500 ohm·cm to about 5000,000 ohm·cm. In some embodiments the oxygen-deficient metal oxide has a resistivity in the range of about 1000 ohm·cm to about 5000,000 ohm·cm. In some embodiments the oxygen-deficient metal oxide has a resistivity in the range of about 5000 ohm·cm to about 5000,000 ohm·cm. In some embodiments the oxygen-deficient metal oxide has a resistivity in the range of about 1000 ohm·cm to about 10000 ohm·cm.

A number of suitable oxygen-deficient metal oxides may be used in the semiconducting portion. In one embodiment the oxygen-deficient metal oxide semiconducting portion may be formed using any suitable metal oxide selected from the group consisting of zinc oxide (ZnO), strontium titanium oxide (STO), tin oxide (SnO₂), and titanium dioxide (TiO₂).

A In a preferred form, the semiconducting portion is an oxygen-deficient metal oxide layer formed using zinc oxide (ZnO) or strontium titanium oxide (STO). As will be described below, the inventors have found that good results may be obtained when the metal oxide layer is a thin film oxygen-deficient zinc oxide (ZnO) layer.

The oxygen-deficient metal oxide semiconducting portion may be applied to the substrate surface by a technique selected from the group consisting of reactive sputtering, physical vapour deposition (PVD), chemical vapour deposition (CVD), metal organic chemical vapour deposition (MOCVD), pulsed laser deposition (PLD) and molecular beam epitaxy (MBE).

In one embodiment, the oxygen-deficient metal oxide layer is applied to the surface of a rigid (SiO₂/Si) wafer or a flexible polyimide foil by reactive sputtering to afford a thin metal oxide film having a thickness that falls within a range of about 50 nm to about 200 μm.

In certain embodiments the semiconducting portion is formed by zinc oxide which has been sputtered onto the surface of a rigid (SiO₂/Si) wafer to provide an oxygen-deficient zinc oxide layer (ZnO₁₋ₓ) that presents a plurality of hydroxy (OH) groups at the surface. The as-deposited oxygen-deficient ZnO layer may be of any suitable thickness to suit the desired application. The applicants have found that good results have been obtained when the oxygen-deficient ZnO layer has a thickness that falls within the range of about 10 nm to about 1 μm.

### Zinc Oxide (ZnO)

Two different types of ZnO thin films with different ratios of oxygen content are prepared via magnetron sputtering. This results in different stoichiometries of the sputtered thin films. The sputtering parameters and associated conductivity are listed in Table 1.

TABLE 1
Parameters used to sputter ZnO thin films with different
stoichiometries and associated electrical conductivity.

| Thin | | | | Processing | Processing | |
|---|---|---|---|---|---|---|
| Film | Sputtering | Power | Ar:O₂ | pressure | Temperature | Conductivity |
| Types | Source | (W) | ratio | (mTorr) | (° C.) | (S/m) |
| ZnOₓ | Zn (metallic) | 200 | 100:30 | 3.5 | 250 | 0.08-0.6* |
| ZnO_{y} | ZnO (ceramic) | 200 | 100:05 | 3.5 | 250 | <0.07** |

*this corresponds to a resistivity of 166 to 1250 ohm · cm
**this corresponds to a resistivity of greater than 1428 ohm · cm

The sputtering parameters are selected to engineer thin films with electrical conductivities in the range of 0.08-0.6 S/m. This range of conductivity gives good sensitivity of the sensors.

### Strontium Titanium Oxide (STO)

Two different types of strontium titanium oxide (SrTiO₃: STO) thin films are prepared via magnetron sputtering with different ratios of oxygen content. The sputtering parameters are summarised in Table 3.

TABLE 3
Parameters used to sputter STO thin films with different
stoichiometries and associated conductivity.

| Thin | | | | Processing | Processing | |
|---|---|---|---|---|---|---|
| Film | Sputtering | Power | Ar:O₂ | pressure | Temperature | Conductivity |
| Types | Source | (W) | ratio | (mTorr) | (° C.) | (S/m) |
| STOₓ | Ceramic SrTiO₃ | 200 | 100:00 | 3.5 | 23 | 0.07-0.09*** |
| STO_{y} | Ceramic SrTiO₃ | 200 | 100:05 | 3.5 | 23 | <0.04**** |

***this corresponds to a resistivity of 1111 to 1428 ohm · cm
****this corresponds to a resistivity of greater than 2500 ohm · cm

### Oligonucleotide

The sensing element of the sensor includes at least one, and typically a plurality of oligonucleotides on a surface of the semiconducting portion. The oligonucleotide is chosen such that it is complementary to the nucleic acid sequence or sequences to be detected. As used herein the term ‘complementary” means that the nucleic acid sequence or sequences to be detected will bind with the oligonucleotide as a result of base pairing throughout substantially the full length of the nucleic acid sequence. In some embodiments, the sensing element includes a plurality of oligonucleotides on the surface of the semiconducting portion.

The oligonucleotide(s) may be immobilised on the semiconducting portion of the substrate by either physical absorption or chemical bonding. In a preferred form, the oligonucleotide is chemically bonded to the surface of the semiconducting portion.

Semiconductors such as oxygen-deficient metal oxides or non-oxide semiconductors, including silicon semiconductors, typically contain surface functionality such as hydroxy groups which are susceptible to covalent bond-forming reactions with surface modification agents such as silanizing agents (surface modification agents containing silanizing groups such as alkoxy silanes). The oligonucleotide may thus be chemically bonded to the semiconducting portion by a process comprising: (i) silanization of the non-oxide semiconductor with a silanizing agent having a terminal functionality selected from the group consisting of an epoxy group, a thiol group, an amino group, a carboxy group and a hydroxy group, and (ii) reacting a suitably functionalised oligonucleotide with the terminal functionality. As a result of this process, the binding site is anchored to the surface of the semiconducting portion by an organic linker, being the residue of the silanizing agent.

Suitable silanizing agents include (3-glycidyloxypropyl)trimethoxysilane (GPS), (3-mercaptopropyl)trimethoxysilane (MTS), (3-aminopropyl)triethoxysilane (APTES), and N-(2-aminoethyl)-3-aminopropyl-trimethoxysilane (AEAPTS), and the like. For example, when an epoxy-functionalised silanizing agent such as (3-glycidyloxypropyl)trimethoxysilane (GPS) is used, silanization of the non-oxide semiconductor functionalises its surface with pendant epoxy groups. A suitably functionalised oligonucleotide such as an amino functionalised oligonucleotide may thus be immobilised on this surface by conjugation reactions of epoxy-reactive functional groups with the amine.

In another set of embodiments, the oligonucleotide is initially present on a molecule which is pre-functionalised with a surface-reactive functional group such as a silanizing group. The oligonucleotide may thus be chemically bonded to the semiconducting portion by contacting the pre-functionalised biomolecule (or other entity) with the semiconducting portion under conditions suitable to allow covalent bond formation and thus surface immobilisation.

In certain embodiments the oligonucleotide is attached to the surface of the semiconducting portion via the 3′ end of the oligonucleotide. In certain embodiments the oligonucleotide is attached to the surface of the semiconducting portion via the 5′ end of the oligonucleotide.

The sensors of the present invention may be used to detect a wide variety of nucleic acids with the only real limitation being that in order to detect a nucleic acid it is necessary to attach to the semiconducting portion with an oligonucleotide that is complementary (as discussed above) to the nucleic acid to be detected. In general, with advances in nucleic acid sequencing and oligonucleotide synthesis this does not cause any significant difficulty as in general the target nucleic acid to be detected has been well characterised as it is typically associated with a medical condition to be diagnosed.

In one embodiment the oligonucleotide is complementary to a nucleic acid sequence having a single point mutation relative to a native DNA sequence. In one embodiment the oligonucleotide is from the human BRAF. In one embodiment the oligonucleotide is from the human KRAS gene. In one embodiment the oligonucleotide is from the human PIK3CA gene. In one embodiment the oligonucleotide is complementary to a nucleic acid having an insertion or deletion mutation relative to a native DNA sequence. In one embodiment the oligonucleotide is from the human EGFR gene. In one embodiment the oligonucleotide is complementary to human micro RNA nucleic acid. In one embodiment the oligonucleotide is from the human micro RNA miR-371a. In one embodiment the oligonucleotide is complementary to a nucleic acid having a common variation in human native DNA sequence. In one embodiment the oligonucleotide is from the human DPYD gene. In one embodiment the oligonucleotide is SEQID No1 or SEQID No2.

The semiconducting portion of the substrate may comprise an oxidic surface layer on the semiconducting portion, the oxidic layer comprising the surface functionality susceptible to covalent bond-forming reactions with surface modification agents. Such passivation layers are generally very thin, so that binding of the oligonucleotide will cause a change to the resistance of the underlying high-resistivity semiconductor in use. Any oxidic surface layer at the surface of the semiconducting portion may thus be less than 10 nm in thickness.

### Detection Method

The present invention also relates to a method for detecting a nucleic acid. The method comprises the steps of (a) contacting a sensing element of a sensor as described herein with a substance possibly containing the nucleic acid, (b) measuring an electrochemical parameter of the sensor corresponding to the resistance of the sensor and (c) detecting the presence or absence of the nucleic acid on the sensing element based on the electrochemical parameter measured in step (b).

The nucleic acid to be detected is typically located in a substance which may be any substance which contains, or may contain, a nucleic acid sequence of interest. In some embodiments, the substance is a sample solution, for example a liquid sample which is, or contains, a bodily fluid such as saliva, sweat, blood or urine, or extracted nucleic acid(s) from tissues or tumours.

As discussed above the method relies on the nucleic acid to be detected hybridising with the oligonucleotide which is located on the sensor and which is complementary to the nucleic acid to be detected. Accordingly, there is a requirement that the sensing element of the invention be bought into contact with the substance for a period of time sufficient for the hybridisation to occur. For example, where the substance is a fluid there is a requirement that the nucleic acid be provided with sufficient time to diffuse through the liquid to the oligonucleotide and hybridise with it.

In principle the time of contact of the sensing element with the substance sample can be any length of time as long as the nucleic acid to be detected is in contact with the substance for sufficient time to hybridise with the oligonucleotide. In certain embodiments the sensing element is contacted with the substance for a period of time from 1 minute to 60 minutes. In certain embodiments the sensing element is contacted with the substance for a period of time from 1 minute to 30 minutes. In certain embodiments the sensing element is contacted with the substance for a period of time from 1 minute to 20 minutes. In certain embodiments the sensing element is contacted with the substance for a period of time from 1 minute to 10 minutes. In certain embodiments the sensing element is contacted with the substance for a period of time from 5 minutes to 10 minutes.

Following contact of the sensing element with the sample the sensing element is typically washed with phosphate buffer solution (PBS, pH 7.4) to remove any substance from the surface of the sensor.

As stated previously the hybridisation of the nucleic acid to the oligonucleotide leads to a change in the resistance of the sensor. Accordingly, following contacting of the sensing element with the substance an electrochemical parameter of the sensor is measured that corresponds to a resistance of the sensor.

As will be appreciated by a skilled worker in the field there are a number of different electrochemical parameters of the sensor that may be measured that correspond to the resistance of the sensor. For example, one could apply a fixed current across the sensor and determine the voltage across the sensor. Alternatively, one could apply a fixed voltage across the sensor and determine the current flow.

In typical operation of a conductometric sensor, the directly measured parameter is the current response when a known voltage (or voltage profile) is applied across the sensor. In some embodiments, therefore, the method of measuring an electrochemical parameter of the sensor comprises (i) applying a voltage across the sensor, and measuring a current flow through the sensor. The voltage may be applied, and the current flow measured, using conventional apparatus for conductometric sensors, such as a potentiostat.

However, it is not excluded that a different electrochemical parameter corresponding to the sensor resistance may be measured. For example, it is possible in principle to pass a predetermined current through the sensor and measure the voltage required to achieve this current. In that case, the measured voltage corresponds to the sensor resistance. In one embodiment an increase in resistance of the sensor in comparison to the reference resistance is indicative of the presence of the nucleic acid in the sample.

The presence or absence of the nucleic acid on the sensing element may be detected by comparing the measured electrochemical parameter with a reference value for that parameter for the sensor. When the measured parameter is a current response, the current flow, or an electrical resistance of the sensor determined from the current flow, to a predefined reference current flow, or resistance, for the sensor corresponding to the presence or absence of the nucleic acid on the sensing element. For example, the current flow (or resistance) of the sensor after contact with the substance may be compared against the current flow (or resistance) of the sensor after contact with a reference solution which does not contain the nucleic acid. In its simplest form, such a comparison may be used to determine the presence or absence of the nucleic acid in the substance. Alternatively, the current flow (or resistance) of the sensor after contact with a sample solution containing the nucleic acid may be compared against a calibration curve which plots the current flow (or resistance) of the sensor after contact with a series of reference solutions having known concentrations of the nucleic acid. In this way, the concentration of the nucleic acid in the sample solution may be calculated.

The method may optionally include one or more preparation steps between the steps of contacting the sensing element with the substance and applying the voltage. For example, when the substance is a sample solution, the sensing element may be incubated for a defined time at defined conditions (e.g. of temperature) to allow binding of the nucleic acid (if present in the sample solution) to the oligonucleotide sites. The sample solution may then be removed from the sensor and the sensing element washed and/or dried before performing the conductometric measurements.

Alternatively, the sensor may be used as an invasive sensor which is inserted into the human body for in situ detection of a nucleic acid, for example when integrated into a microneedle. In another embodiment, the sensor is integrated into a wearable device for monitoring a nucleic acid in human sweat.

In principle the sensor of the present invention may be used to detect the presence of any nucleic acid of interest. Indeed, whilst the sensor has been exemplified in respect of the BRAF and cancer associated sequences in principle the senor can be used to detect any nucleic acid. The only limitation on the ability of the sensor to detect nucleic acid sequences.

### Method of Fabricating a Sensor

The invention also relates to a method of fabricating a sensor for detecting a nucleic acid. The method includes a step of providing a substrate which comprises a semiconducting portion. A pair of terminal electrodes is produced on the substrate in mutually spaced apart and opposing relation such that the semiconducting portion of the substrate is positioned between and in electrical contact with the terminal electrodes and a conduction path between the terminal electrodes passes through the semiconducting portion. An oligonucleotide is then immobilised on a surface of the semiconducting portion, thereby producing a sensing element comprising (i) the semiconducting portion and (ii) an oligonucleotide on the surface of the semiconducting portion.

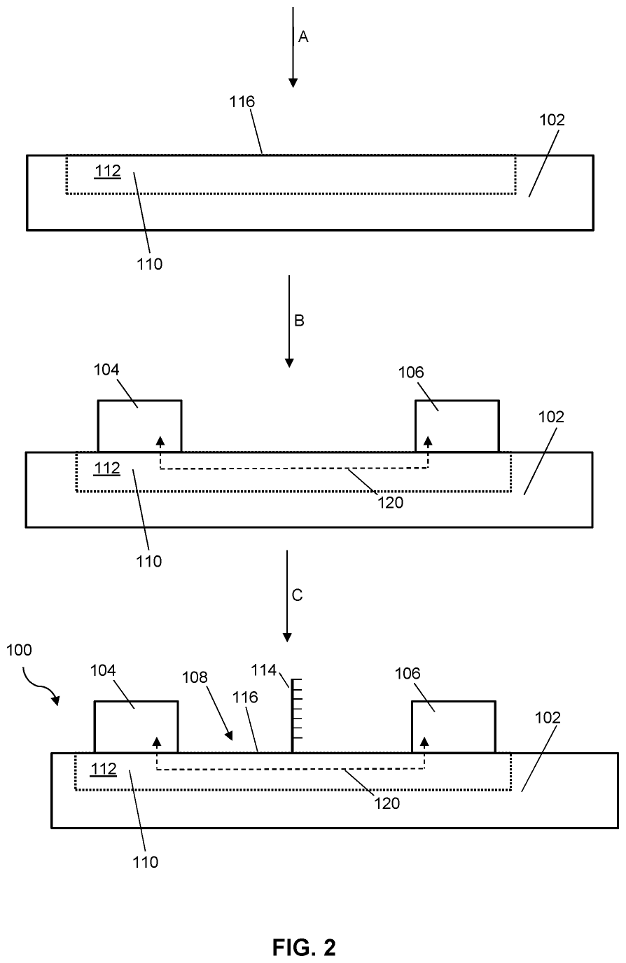

In one form of the invention, as shown in the schematic representation in FIG. 2, substrate 102 is provided in step A. Substrate 102 includes semiconducting portion 110 which comprises a semiconducting material 112 as described herein. In the embodiment shown in FIG. 2, substrate 102 comprises semiconducting portion 110 as an integral part of the substrate, and the remainder of the substrate is thus composed of the same semiconducting material 112. Alternatively, substrate 102 may include semiconducting portion 110 formed as a discrete, thin surface layer on an underlying support layer, which may be composed of any suitable material capable of receiving and supporting semiconducting layer 110.

In step B, a pair of terminal electrodes 104, 106 is produced on substrate 102 in mutually spaced apart and opposing relation. The electrodes are produced such that semiconducting portion 110 of the substrate is positioned between and in electrical contact with terminal electrodes 104, 106. A conduction path 120 between terminal electrodes 104 and 106 thus passes through semiconducting portion 110, and therefore also through the semiconducting material 112.

In step C, oligonucleotide 114 is immobilised on surface 116 of the semiconducting portion, thereby producing sensing element 108. While FIG. 1 depicts a single binding site, it will be appreciated that a plurality of oligonucleotides 114 may be immobilised on surface 116. Sensing element 108 comprises semiconducting portion 110 and the oligonucleotide(s) 114. Sensor 100, as previously described herein with reference to FIG. 1, is thus fabricated after performing steps A, B and C.

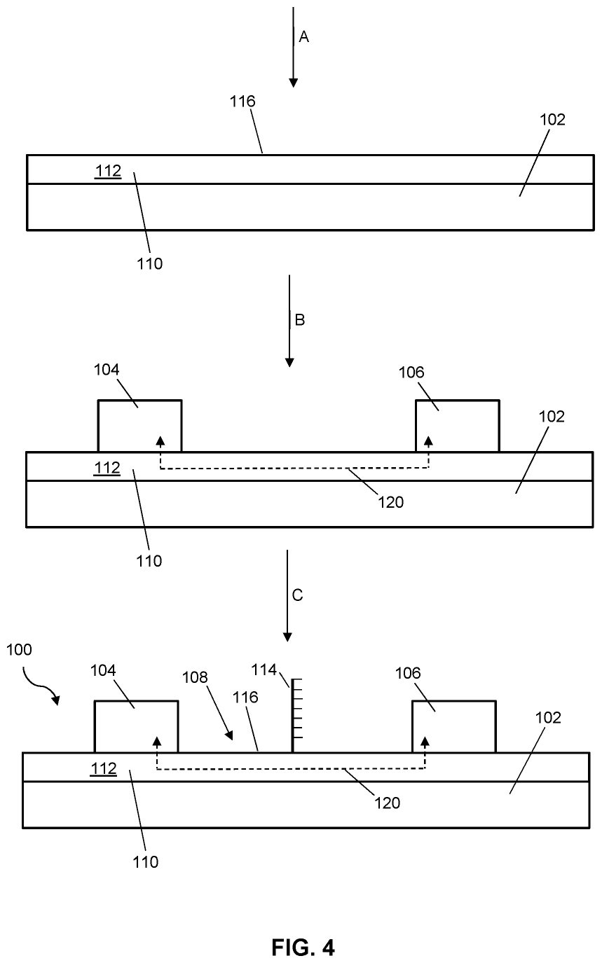

In another form of the method of fabrication of the invention, as shown in the schematic representation in FIG. 4, substrate 102 is provided in step A. Substrate 102 includes semiconducting portion 110 which is present as a layer 112 of semiconducting material as described herein. In the embodiment shown in FIG. 4, substrate 102 comprises a semiconducting portion 110 formed as a discrete, thin surface layer 112 on an underlying support layer, which may be composed of any suitable material capable of receiving and supporting semiconducting layer 110.

In step B, a pair of terminal electrodes 104, 106 is produced on substrate 102 in mutually spaced apart and opposing relation. The electrodes are produced such that semiconducting portion 110 of the substrate is positioned between and in electrical contact with terminal electrodes 104, 106. A conduction path 120 between terminal electrodes 104 and 106 thus passes through semiconducting portion 110, and therefore also through semiconducting material 112.

In step C, oligonucleotide 114 is immobilised on surface 116 of the semiconducting portion, thereby producing sensing element 108. While FIG. 4 depicts a single oligonucleotide, it will be appreciated that a plurality of oligonucleotide sites 114 may be immobilised on surface 116. Sensing element 108 comprises semiconducting portion 110 and the oligonucleotide (s) 114. Sensor 100, as previously described herein with reference to FIG. 3, is thus fabricated after performing steps A, B and C.

The substrate comprising a semiconducting portion may be according to any of the embodiments described herein in the context of the sensors of the invention.

The terminal electrodes may be produced on the substrate by any suitable method. In some embodiments, the terminal electrodes are formed by microfabrication techniques. Gold terminal electrodes may be formed by evaporating a gold thin film (250 nm with 100 nm chromium adhesion layer) onto the semiconducting layer using electron beam lithography. The as deposited gold thin film is then patterned using standard photolithography and wet etching techniques to define the pair of terminal electrodes.

The oligonucleotide binding sites may be immobilised on the surface of the semiconducting portion by either physical absorption or chemical bonding. In a preferred form, the oligonucleotide(s) are chemically bonded to the surface of the semiconducting portion.

The materials used in the semiconducting portion of the substrate of the present invention such as non-oxide semiconductors, including silicon semiconductors or oxygen-deficient metal oxides, typically contain surface functionality such as hydroxy groups which are susceptible to covalent bond-forming reactions with surface modification agents such as silanizing agents (surface modification agents containing silanizing groups such as alkoxy silanes). The oligonucleotide may thus be chemically bonded to the semiconducting portion by a process comprising: (i) silanization of the semiconducting portion with a silanizing agent having a terminal functionality selected from the group consisting of an epoxy group, a thiol group, an amino group, a carboxy group and a hydroxy group, and (ii) reacting a precursor comprising the oligonucleotide with the terminal functionality. As a result of this process, the oligonucleotide is anchored to the surface of the semiconducting portion by an organic linker, being the residue of the silanizing agent.

Suitable silanizing agents include (3-glycidyloxypropyl)trimethoxysilane (GPS), (3-mercaptopropyl)trimethoxysilane (MTS), (3-aminopropyl)triethoxysilane (APTES), and N-(2-aminoethyl)-3-aminopropyl-trimethoxysilane (AEAPTS), and the like.

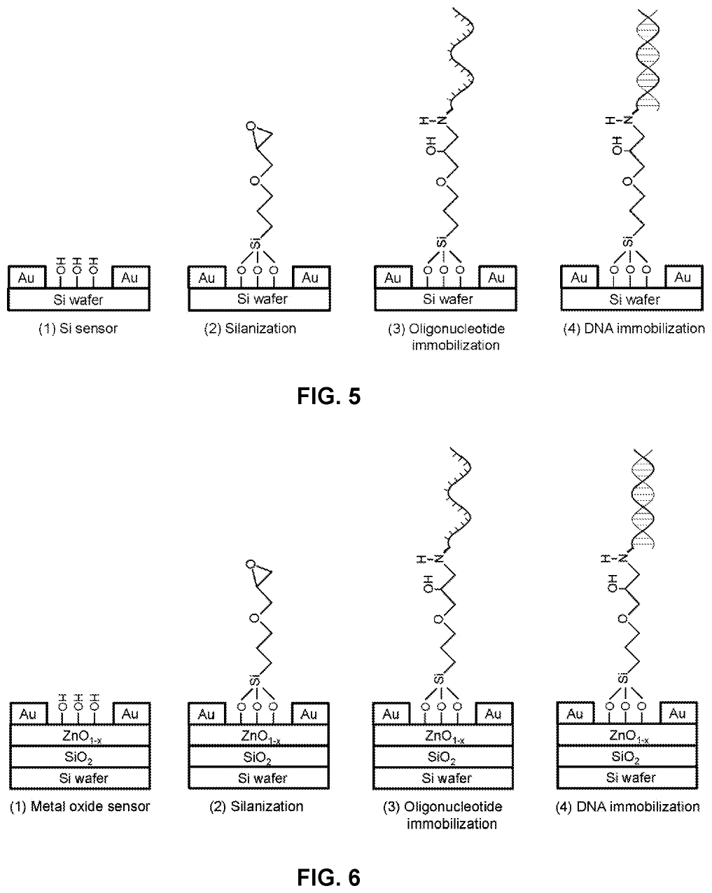

As shown in the schematic representation in FIG. 5 (1), there is provided a semiconducting portion of a substrate, beneath and between gold (Au) terminal electrodes, comprises a semiconducting material, in this case a high-resistivity intrinsic silicon wafer. In step (2) the surface of the semiconducting portion is contacted with silanizing agent, which may optionally be an epoxy-functionalised silanizing agent such as (3-glycidyloxypropyl)trimethoxysilane (GPS). The silanizing agent reacts with surface hydroxy (—OH) functionalities of the semiconducting portion, thus anchoring the silanizing agent to the surface via covalent bonds and functionalising the surface with pendant conjugating groups, in this case epoxy groups. In step (3) an oligonucleotide, is then immobilised on the surface by conjugation reactions of epoxy-reactive functional groups present in the oligonucleotide, in this case an amine (—NH₂). The oligonucleotide is thus anchored to the surface of the semiconducting portion by an organic linking group, which is the residue of the silanizing agent. In use, as shown in step 4, the nucleic acid hybridises with the oligonucleotide to form an immobilised nucleic acid molecule on the sensor.

The same process steps for an alternative embodiment is shown in the schematic representation in FIG. 6. In FIG. 6 (1), there is provided a sensor where the semiconducting portion of a substrate is provided as a layer on a support portion of a substrate, beneath and between gold (Au) terminal electrodes. The layer shown is an oxygen-deficient zinc oxide layer. In step (2) the surface of the semiconducting portion is contacted with silanizing agent, which may optionally be an epoxy-functionalised silanizing agent such as (3-glycidyloxypropyl)trimethoxysilane (GPS). The silanizing agent reacts with surface hydroxy (—OH) functionalities of the semiconducting portion, thus anchoring the silanizing agent to the surface via covalent bonds and functionalising the surface with pendant conjugating groups, in this case epoxy groups. In step (3) an oligonucleotide, is then immobilised on the surface by conjugation reactions of epoxy-reactive functional groups present in the oligonucleotide, in this case an amine (—NH₂). The oligonucleotide is thus anchored to the surface of the semiconducting portion by an organic linking group, which is the residue of the silanizing agent. In use, as shown in step 4, the nucleic acid hybridises with the oligonucleotide to form an immobilised nucleic acid molecule on the sensor.

### EXAMPLES

### Materials and Methods

High-resistivity silicon wafer (100 mm diameter) with resistivity of 1000-2000 ohm·cm was purchased from D & X Co. Ltd., Japan and it was single side polished silicon wafer. The orientation of 1000-2000 ohm·cm wafer was <100> and the thickness was 500±10 μm.

Silicon wafer sensors were fabricated by patterning two terminal in-plane electrodes on the high-resistivity silicon wafers using standard photolithography processes. The electrode gap could be in the range from 1-2 μm to 100 μm. However, this electrode gap was optimised to be 40 μm for the best sensor performance. The length of the electrodes was in the range from 200 μm to 4000 μm. The optimum electrode length was set to 4000 μm. The sensing element area (silicon substrate area between the electrodes) was 16×10⁻⁸ m². Similar specifications were observed for metal oxide layers.

The conductance of sensors was measured using a commercial current source meter (B2901A precision source/measure unit from Keysight Technologies). The sensors were placed on a LTS120 Linkam Stage as a sensor holder in all measurements. Keysight Quick I-V Measurement software was used in data acquisition. The bias across the electrodes was maintained at 1.8 V. The resistance measurements on sensors were acquired after oligonucleotide immobilisation and after complementary DNA hybridisation. The data acquisition time for a given sensor was 1 min.

The wild-type DNA (NA 12878) in PBS and melanoma A375 DNA with point mutant V600E in PBS were provided by Peter MacCallum Cancer Centre and used as-received.

### Example 1. Preparation of GPS-Silanized Silicon Wafer Sensors

Silicon wafer sensors were fabricated by patterning two terminal in-plane electrodes on the high-resistivity silicon wafers using standard photolithography processes. The electrode gap could be in the range from 1-2 μm to 100 μm. However, this electrode gap was optimised to be 40 μm for the best sensor performance. The length of the electrodes was in the range from 200 μm to 4000 μm. The optimum electrode length was set to 4000 μm. The sensing element area (silicon substrate area between the electrodes) was 16×10⁻⁶⁸ m².

Silanization of the silicon wafer sensor surfaces using (3-glycidyloxypropyl)trimethoxy silane (GPS) was conducted after exposing freshly prepared sensor devices to O₂ plasma for 10 minutes (Plasma Cleaner PDC-002, Harrick Plasma) to activate the hydroxyl groups on the silicon surface. Then, 20 μL of freshly prepared GPS solution was drop-cast onto an Al foil, which was placed inside a vacuum desiccator, allowing GPS vapor to build up inside the desiccator. Then, the O₂ plasma cleaned-silicon sensors were exposed to this GPS vapor for 30-45 min inside an LC 200 Glovebox System. Afterwards, the silanized silicon wafer sensors were rinsed thoroughly with Milli-Q water for 2 minutes to remove any unbound silane groups from the surface. Then, the washed sensors were heated at 150° C. for 10 minutes to strengthen the bonding of the silane groups to the silicon wafer surface. These GPS-silanized silicon wafer sensors, which are functionalised with surface epoxide functional groups chemically bonded to the substrate surface, were then used to immobilize oligonucleotides.

### Example 2. Preparation of GPS-Silanized Zinc Oxide Wafer Sensors

The sensors were fabricated by following standard micro-nano-fabrication techniques on rigid (50-300 nm SiO₂/500 μm Si) and flexible plastic (polyimide foils, 75-125 μm thick) substrates by depositing a 80-100 nm thick thin film of a metal oxide, such as oxygen-deficient zinc oxide (ZnO) acting as sensing layer in the biosensors. The composition of sensing layer is fabricated by reactive sputtering to produce an oxygen deficient metal oxide film with conductance in the range of 1-2 siemens/m. For conductance measurements, two terminal in-plane electrodes are patterned and fabricated with a sensing area of 16×10⁻⁸ m².

Freshly prepared ZnO devices were exposed to O₂ plasma for 10 min (commercial Plasma Cleaner PDC-002, Harrick Plasma) to clean the device surface from organic contaminants and to activate the hydroxyl groups on ZnO surface. Then 20 μL of freshly prepared GPS solution was drop casted on a commercial Al foil which was placed inside a vacuum desiccator, allowing to build GPS vapor inside the desiccator. Then the O₂ plasma cleaned-ZnO sensors were exposed to this GPS vapor for 1-2 h. The exposure of ZnO sensors to GPS vapor was conducted inside the LC 200 Glovebox System. Upon the completion of exposure to GPS vapor, the ZnO sensors were rinsed thoroughly with Mili-Q water for 2 min to remove the unbounded silane groups from ZnO devices. Then the washed ZnO sensors were heated at 150° C. for 10 min to strengthen the bonding of silane groups onto ZnO surface. These GPS-silanized sensors were used for the immobilization of oligonucleotides.

### Example 3. Immobilisation of Oligonucleotides

This example exemplifies the immobilization of the oligonucleotide on a ZnO sensor of example 2. Similar methodology is used to immobilize the oligonucleotide on other sensors.

BRAF 3′-amine DNA oligonucleotide (5′-GGTCTAGCTACAGAGAAATCTCGAT/3AmMO/-3′) and BRAF 5′-amine DNA oligonucleotide (5′-/AmMC6/GGTCTAGCTACAGAGAAATCTCGAT-3′) were purchased from Integrated DNA Technologies, Inc., USA and used as-received. A 15 μL volume of freshly prepared 35 pM oligonucleotide solution (prepared in pH 7.4 phosphate buffer saline (PBS)) was drop casted on each freshly GPS-silanized ZnO sensors and incubated for 1 h allowing the oligonucleotides to immobilize on the ZnO sensors. Then the sensors were rinsed extensively with pH 7.4 PBS solution to remove the unbounded oligonucleotides. The PBS-washed ZnO sensors were then dried under N₂ gas for 2 min prior to the conductometric measurements.

### Example 4. Conductometry of DNA-ZnO Sensor

The wild-type DNA (NA 12878) in PBS and melanoma A375 DNA with point mutant V600E in PBS were provided by Peter MacCallum Cancer Centre and used as-received. The baseline conductance of the oligonucleotide-immobilized ZnO sensors was measured prior to the addition of DNA samples. A 15 μL volume of the DNA solution (wild-type DNA or point mutant DNA) was drop casted on the oligonucleotides-immobilized ZnO sensors and incubated for 10 min. After 10 min, the remaining DNA solution on the sensor was removed and the surface was dried under N₂ gas followed by conductance measurements. For the cross-selective measurements, a series of DNA mixtures were prepared by pre-mixing the two types of DNA samples in the volume ratios of (point mutant DNA:wild-type DNA) 1:200, 1:100, 1:1, 100:1, and 200:1. As the molar concentrations of stock DNA samples are the same, the volume ratio of the two DNA types is identical to the molar ratio of the two DNA types.

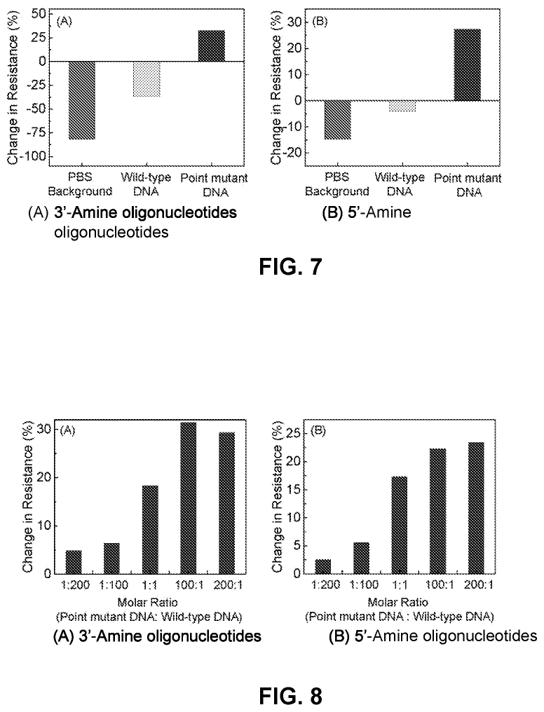

Both 3′-amine and 5′-amine oligonucleotides displayed a reversal of polarity in resistance change upon hybridization with point mutant DNA strands compared to the resistance change obtained for hybridization with wild-type DNA strands (FIG. 7). The change in resistance is the percentage change of resistance of the device after DNA hybridization with respect to the baseline resistance. The baseline resistance is the resistance of the oligonucleotide immobilized-device prior to the DNA addition. The resistance of the device increased upon hybridization with point mutant DNA while the resistance of the device decreased after hybridization with wild-type DNA. This result suggests that point mutant DNA acts as an electron acceptor and wild-type DNA acts as an electron donor upon their hybridizations with both types of oligonucleotides. The reverse polarity in change in resistance for point mutant DNA highlights the high sensitivity of these conductometric devices to detect cancer DNA such as melanoma with a single base pair different (V600E variant) compared to the healthy DNA. The absolute change in resistance values obtained for all the samples tested are higher in 3′-amine oligonucleotides immobilized-devices than 5′-amine oligonucleotides immobilized-devices. This indicates that the charge transfer process is much more feasible on 3′-amine oligonucleotides than 5′-amine oligonucleotides. The resistance change for PBS solvent is in the same polarity with wild-type DNA and not with point mutant DNA. As such, the contribution from the matrix of the point mutant DNA solution for the change in resistance is negligible.

### Example 5. Cross Selectivity Study

To determine the viability of the conductometric devices for the detection of V600E point mutant DNA in the presence of other DNA, cross-selectivity measurements were also conducted. Both types of oligonucleotides immobilized-devices selectively detected V600E point mutant DNA in the presence of wild-type DNA (FIG. 8). The resistance change was increased with the increased V600E point mutant DNA fraction and reached a saturation value beyond the molar fraction of point mutant DNA: wild-type DNA was 100:1. This trend is seen in both types of oligonucleotides. Both oligonucleotides successfully detected V600E point mutant DNA at point mutant DNA: wild-type DNA molar ratio of 1:200 which is corresponding to the clinically important allele fraction ratio (i.e. 0.5%). This result highlights high sensitivity and high selectivity of these conductometric sensors in V600E point mutant DNA detection.

### Example 6. Conductometry of DNA on a Si Sensor

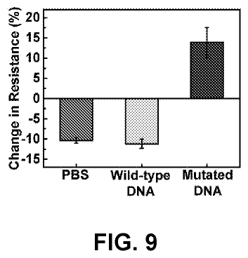

An experiment was conducted using the methodology of example 4 but using a silicon sensor of example 1 with immobilised with 5′-amine oligonucleotides. The results of the conductometric experiments are shown in FIG. 9.

### Definitions

Whenever a range is given in the specification, for example, a temperature range, a time range, or concentration range, all intermediate ranges and subranges, as well as all individual values included in the ranges given are intended to be included in the disclosure. It will be understood that any subranges or individual values in a range or subrange that are included in the description herein can be excluded from the claims herein.

All definitions, as defined and used herein, should be understood to control over dictionary definitions, definitions in documents incorporated by reference, and/or ordinary meanings of the defined terms.

The indefinite articles “a” and “an,” as used herein in the specification, unless clearly indicated to the contrary, should be understood to mean “at least one.”

The phrase “and/or,” as used herein in the specification, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

While the invention has been described in conjunction with a limited number of embodiments, it will be appreciated by those skilled in the art that many alternatives, modifications and variations in light of the foregoing description are possible. Accordingly, the present invention is intended to embrace all such alternatives, modifications and variations as may fall within the spirit and scope of the invention as disclosed.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components, or group thereof.

1. A sensor for detecting a nucleic acid, comprising:
a substrate;
a pair of terminal electrodes disposed on the substrate in mutually spaced apart and opposing relation; and
a sensing element, between and in electrical contact with the pair of terminal electrodes, wherein the sensing element comprises:
(i) a semiconducting portion of the substrate, wherein a conduction path between the terminal electrodes passes through the semiconducting portion; and
(ii) an oligonucleotide on a surface of the semiconducting portion, the oligonucleotide being complementary to the nucleic acid to be detected,
wherein hybridisation of the nucleic acid with the oligonucleotide leads to a change in resistance of the sensor.

2. The sensor according to claim 1, wherein the semiconducting portion has a resistivity of greater than 100 ohm·cm.

3. (canceled)

4. The sensor according to claim 1, wherein the semiconducting portion has a resistivity in the range of about 1000 ohm·cm to about 10000 ohm·cm.

5. The sensor according to claim 1, wherein the semiconducting portion comprises a high-resistivity non-oxide semiconductor, and wherein the non-oxide semiconductor has a resistivity of greater than 100 ohm·cm.

6-9. (canceled)

10. The sensor according to claim 5, wherein the non-oxide semiconductor is an intrinsic silicon semiconductor.

11. (canceled)

12. The sensor according to claim 10, wherein the silicon semiconductor is a float-zone silicon semiconductor.

13-14. (canceled)

15. The sensor according to claim 1, wherein the semiconducting portion comprises an oxygen deficient metal oxide, and wherein the oxygen-deficient metal oxide is selected from the group consisting of zinc oxide (ZnO), strontium titanium oxide (STO), tin oxide (SnO₂), and titanium dioxide (TiO₂).

16-18. (canceled)

19. The sensor according to claim 1, wherein the oligonucleotide is chemically bonded to the semiconducting portion by a process comprising: (i) silanization of the semiconducting portion with a silanizing agent having a terminal functionality selected from the group consisting of an epoxy group, a thiol group, an amino group, a carboxy group and a hydroxy group, and (ii) reacting an oligonucleotide with the terminal functionality.

20. (canceled)

21. The sensor according to claim 1, wherein the oligonucleotide is complementary to a nucleic acid having a single point mutation to a native DNA sequence.

22-23. (canceled)

24. The sensor according to claim 1, wherein the oligonucleotide is the BRAF oligonucleotide.

25. (canceled)

26. A method for detecting a nucleic acid, the method comprising the steps of:
a) contacting a sensing element of a sensor according to claim 1 with a substance possibly containing the nucleic acid;
b) measuring an electrochemical parameter of the sensor corresponding to a resistance of the sensor; and
c) detecting the presence or absence of the nucleic acid on the sensing element based on electrochemical parameter measured in step (b).

27-35. (canceled)

36. A method of fabricating a sensor for detecting a nucleic acid the method comprising the steps of:
a. providing a substrate comprising a semiconducting portion;
b. producing a pair of terminal electrodes on the substrate in mutually spaced apart and opposing relation, wherein the semiconducting portion of the substrate is positioned between and in electrical contact with the terminal electrodes and wherein a conduction path between the terminal electrodes passes through the semiconducting portion; and
c. immobilizing an oligonucleotide on a surface of the semiconducting portion, the oligonucleotide being complementary to the nucleic acid to be detected, thereby producing a sensing element comprising (i) the semiconducting portion and (ii) the oligonucleotide.

37. The method according to claim 36, wherein the semiconducting portion has a resistivity of greater than 100 ohm·cm.

38-52. (canceled)

53. The method according to claim 36, wherein the oligonucleotide is chemically bonded to the semiconducting portion.

54. The method according to claim 53, wherein the oligonucleotide is chemically bonded to the semiconducting portion by a process comprising: (i) silanization of the semiconducting portion with a silanizing agent having a terminal functionality selected from the group consisting of an epoxy group, a thiol group, an amino group, a carboxy group and a hydroxy group, and (ii) reacting an oligonucleotide with the terminal functionality.

55-60. (canceled)


SEQUENCE LISTING (2 sequences)

SEQ ID NO: 1 - DNA, 25 bp, Artificial Sequence
  Synthetic DNA probe
  3′ Amino Modifier C3 (25)..(25)
ggtctagctacagagaaatctcgat

SEQ ID NO: 2 - DNA, 25 bp, Artificial Sequence
  Synthetic DNA probe
  5′ Amino Modifier C6 (1)..(1)
ggtctagctacagagaaatctcgat